(19) United States
(12) Patent Application Publication
(10) Pub. No.: US 2005/0095277 A1
(43) Pub. Date: May 5, 2005

(54) Neuropathy cream

(76) Inventors: Binnur Ozturk, (US); Ahmet H. Ozturk, (US)

Correspondence Address: JENNIFER MEREDITH;MEREDITH & KEYHANI, 81 LINWOOD AVE, BUFFALO, NY, 14209, US

(21) Appl. No.: 11/013,072

(22) Filed: Dec. 15, 2004

Related U.S. Application Data
(63) Continuation-in-part of application No. 10/603,311, filed on Jun. 25, 2003.

(51) Int. Cl. A61K 31/195; A61K 31/16; A61L 15/16

(52) U.S. Cl. 424/448; 514/561

(57) ABSTRACT

The present invention provides compositions for transdermal pain relief. One composition for pain relief is comprised of between 1% and 20% by weight ketamine, between 0.2% and 0.5% by weight clonidine, between 7% and 12% by weight gabapentin. Another comprosition is comprised of between 1% and 20% by weight Ketamine, 0.2% and 0.5% by weight clonidine, and 1% and 5% by weight amitriptyline.

### BACKGROUND OF THE INVENTION

This application is a continuation in part of co-pending application Ser. No. 10/603,311 filed Jun. 25, 2003.

The present invention relates to methods for treating or preventing pain via topical formulations that induce a local-anesthetic effect when applied to intact skin.

Pain results from the noxious stimulation of nerve endings. Nociceptive pain is caused by noxious stimulation of nociceptors (e.g., a needle stick or skin pinch), which then transmit impulses over intact neural pathways to the spinal neurons and then to the brain. Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed). Generally, such damage can be caused by a variety of means including trauma, diseases such as diabetes, herpes zoster and late-stage cancer, chemotherapy, or by a chemical injury. Peripheral nerve damage can lead to pathological states where there is a reduction in pain threshold (i.e., allodynia), an increased response to noxious stimuli (hyperalgesia), or an increased response duration (persistent pain).

In the past, patients were generally treated by administration of analgesics to relieve pain. A vast majority of such patients receive doses of these agents orally. Unfortunately, in some situations, oral administration of such agents has been associated with a variety of side effects, such as liver damage, kidney damage, gastrointestinal side effects, addiction, sedation, and/or weight gain which cannot be tolerated well by the patient. In other cases, malabsorption of oral preparations have resulted in subtherapeutic plasma levels. In other cases, the agents have relatively short plasma half-lives, necessitating inconveniently frequent dosing. In general, oral delivery involves a time delay as the analgesic is absorbed via the digestive system before entering the bloodstream. A number of agents which have traditionally been administered orally or by injection have been inappropriate or suboptimal for some patients when so-administered. There are a number of medications which, in at least some patients, are not tolerated well when orally administered (e.g. which cause undesirable gastrointestinal or other side effects) and/or which provide undesirably high or low concentrations or delayed concentrations in a target tissue.

As an alternative to oral preparations, pain can be treated locally by topically administering a local anesthetic directly to the painful area to block the nociceptive mechanistic pathway. Local anesthetics prevent the generation and conduction of nociceptive nerve impulses. Thus, for example, a local anesthetic can be injected intradermally (non-systemic injection within the skin) or topically applied at the pain area. Advantages of topical local-anesthetic administration over systemic administration of pain relievers include decrease or preclusion of side effects, improved patient compliance, and reversible action (i.e., the action can be reversed by removing the anesthetic from the application site). TRANSDERMAL AND TOPICAL DRUG DELIVERY SYSTEMS 33-112 (Tapash K. Ghosh et al. eds., 1997).

A variety of drug classes have local-anesthetic properties and can be administered in topical formulations. Traditional local anesthetics or sodium-channel blockers, such as lidocaine prevent the generation and conduction of nerve impulses by decreasing or preventing the large transient increase in the permeability of excitable membranes to Na+. Other agents with local-anesthetic properties include analgesics, such as non-steroidal anti-inflammatories (“NSAIDs”). N-methyl-D-aspartate (“NMDA”) receptor antagonists, such as ketamine have local-aesthetic properties and topical administration is as an effective neuropathic pain treatment. See, for example, U.S. Pat. No. 5,817,699 (issued Oct. 6, 1998). In another example, topical administration of antidepressant medications, such as amitriptyline, has been reported effective for neuropathic pain treatment. See, for example, U.S. Pat. No. 6,211,171 (issued Apr. 3, 2001); J. Sawynok et al., 82 PAIN 149 (1999). In addition, topical administration of a combination of a tricyclic antidepressant and an NMDA-receptor antagonist is reported to have excellent local-anesthetic properties when topically applied and is useful for treatment of neuropathic pain, U.S. Pat. No. 6,197,830 (issued Mar. 6, 2001).

But even though topical local-anesthetic administration to intact skin is routinely used to treat minor indications, it has not found significant use for treating more severe nociceptive and neuropathic pain because it is difficult to get significant concentrations through the skin barrier. Because of the skin's drug-permeation resistance, as little as about 1 percent and usually no more than about 15 percent of a drug in a topical formulation is bioavailable (TRANSDERMAL AND TOPICAL DRUG DELIVERY SYSTEMS 7 (Tapash K. Ghosh et al. eds., 1997)). Another problem with topical administration of pain relievers is stability of the composition. Local-anesthetics emulsion compositions are inherently unstable, and phase separation can occur during shipment and storage. Furthermore, many topical local-anesthetic compositions suffer from oxidative instability. Lecithin compositions are routinely used as bases for topical local-aesthetic compositions, but are highly oxidatively unstable (AM. PHARM. ASSOC., HANDBOOK OF PHARMACEUTICAL EXCIPIENTS 292-294, 292 (Arthur H. Kibbe ed., 3rd ed. 2000)). For example, U.S. Pat. No. 6,197,830 (issued Mar. 6, 2001) describes a lecithin-based composition for topically administering a combination of an NMDA-receptor antagonist and a tricyclic antidepressant and U.S. Pat. Nos. 5,817,699 (issued Oct. 6, 1998) and 6,017,961 (issued Jan. 25, 2000) describe topical administration of ketamine in pluronic lecithin organogel.

While topical local-anesthetic administration has advantages over systemic administration of pain relievers, they suffer from instability and poor skin-penetration properties, which limit their use to less severe pain. What are needed are stable, effective topical local-anesthetic compositions with good skin-penetration properties the avoid or reduce undesired effects such as liver damage or gastrointestinal side effects.

### SUMMARY OF THE INVENTION

The present invention provides a transdermal composition for the treatment of pain in a subject, particularly a human subject.

According to a preferred embodiment, a transdermal composition for the relief of pain in a subject is disclosed comprising: ketamine, clonidine and gabapentin in a base.

According to another embodiment, a transdermal composition for the relief of pain in a subject is disclosed comprising: ketamine, clonidine and amitriptyline in a base.

According to another embodiment, a transdermal composition for the relief of pain in a subject is disclosed comprising: ketamine and clonidine in a base. This embodiment may be also have ketoprofen.

Another embodiment provides a transdermal composition for the relief of pain in a subject comprising: ketamine in a base. This embodiment may also have clonidine and ketoprofen.

According to yet another embodiment, a transdermal composition for the relief of pain in a subject is disclosed comprising clonidine in a base. This embodiment may also have ketoprofen and ketamine.

These and other features, aspects and advantages of the present invention will become better understood with reference to the following drawings, description and claims.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention provides a transdermal composition suitable for the treatment of pain in a subject. This may include neuropathic pain of all origins including shingles, post-herpetic neuralgia, diabetic neuropathy, peripheral neuropathies, intercostals neuralgia, neuralgias of the trunk and extremities. Also, the present invention may be used to treat arthritis pain, osteoarthritis, rheumatoid arthritis and other arthritic conditions. It may also be used to treat sprains, strains, fibromyalgia, muscular headaches and tension type headaches.

As used herein the term “subject” includes mammals including humans, pigs, cows, mice, rats, rabbits, goats and the like. The preferred subject is a human. As used here, the term “transdermal” composition includes compositions capable of passing through the stratum corneum of a subject. The term transdermal further includes compositions capable passing through the epidermis of a subject, compositions capable of passing through the dermis of a subject, and compositions capable of passing through the hydodermis of subject. In preferred embodiments, the term transdermal includes compositions capable of passing through the skin of a subject and reaching the underlying tissues and organs.

According to one embodiment, a transdermal composition for the relief of pain in a subject is disclosed comprising: ketamine, clonidine and gabapentin. The ketamine would be 1 to 20% by weight, the clonidine 0.2 to 0.5% by weight, and the gabapentin would be 7 to 12% by weight. The base may be any pharmaceutically acceptable carrier which is capable of transdermal delivery of the compounds contained within the composition. This may include a variety of finite and non-finite carriers including liquids, semi-liquids or solid carriers, such as bioadhesives. By way of example, this may be creams, gels, emulsions, lotions, salves, paste, plaster, ointment, spray solution, lipids, phospholipids, lecithins, fatty oils, lanolin, vasoline, paraffins, glycols, higher fatty acids and higher alcohols. Bioadhesive bases may be natural or synthetic polysaccharides. There may also be additives including binders, stabilizers, preservatives, flavorings, fiances, and pigments.

Each of the disclosed embodiments may be in a base. According to a preferred embodiment, the base may be pentravan gel. Pentravan gel is an emollient which softens and moisturizes the skin. Emollients may be used as lubricants to treat or prevent dry, itchy skin and minor skin irritations. The base may also be a base comprised of approximately 80% by weight pleurinic gel and approximately 20% by weight lipoil. The proper base is extremely important, as it acts as a vehicle that allows the various drug components to penetrate the skin. Traditional cream or ointment bases would not be effective. The composition may also comprise 5% to 10% by weight lidocaine. There may also be EMLA. EMLA is a local anesthetic usually used to numb the skin to pain from injections. It is also sometimes used to reduce pain associated with tattooing, electrolysis, laser hair removal, etc. It is generally comprised of lidocaine and prilocaine.

According to another embodiment, there may be a transdermal composition for the relief of pain in a subject comprising: ketamine, clonidine and amitriptyline. The ketamine would be between 1% and 20% by weight, the clonidine between 0.2% and 0.5% by weight, and the amitriptyline may be between 1% and 5% by weight, preferably 4%. Amitriptyline is a tricyclic antidepressant with proven efficacy on neuropathic pain when administered orally. However, oral admininistration has many serious side effects, especially in the elderly population. This may include anticholinergic effects, such as tachycardia, dry mouth and severe sedation. Clonidine is an antihypertensive medicine. Ketamine is generally administered by IV for general anesthesia. It has NMDA inhibitor properties and is effective in controlling pain at spinal cord level.

According to another embodiment, a transdermal composition for the relief of pain in a subject is disclosed comprising: ketamine, clonidine and ketoprofen in a base. The ketamine may be between 1% and 20% by weight, the clonidine may be between 0.2% and 0.5% by weight and the ketoprofen may be between 1% and 5% by weight.

According to another embodiment, a transdermal composition for the relief of pain in a subject is disclosed comprising: ketamine and ketoprofen in a base. The ketamine may be between 1% and 20% by weight and the ketoprofen may be between 1% and 5% by weight.

The present invention also envisions a transdermal composition for the relief of pain in a subject comprising: ketamine and clonidine. The ketamine may be between 1% and 20% by weight and the clonidine may be between 0.2% and 0.5% by weight.

Another embodiment provides a transdermal composition for the relief of pain in a subject comprising: clonidine and ketoprofen in a base. The clonidine may be between 0.2% and 0.5% by weight and the ketoprofen may be between 1% and 5% by weight.

According to yet another embodiment, a transdermal composition for the relief of pain in a subject comprising: ketamine in a based. The ketamine may be between 1% and 20% by weight.

According to yet another embodiment, a transdermal composition for the relief of pain in a subject is disclosed comprising: clonidine in a base. The clonidine may be between 0.2% and 0.5% by weight.

It should be understood that the foregoing relates to preferred embodiments of the invention and that modifications may be made without departing from the spirit and scope of the invention as set forth in the following claims.

1. A transdermal composition for the relief of pain in a subject comprising:
ketamine, clonidine and gabapentin in a base.

2. A transdermal composition as in claim 1, wherein said base is pentravan gel.

3. A transdermal composition as in claim 1, wherein said base is 80% by weight pleurinic gel and 20% by weight lipoil.

4. A transdermal composition as in claim 1, wherein said ketamine is between 1% and 20% by weight.

5. A transdermal composition as in claim 1, wherein said clonidine is between 0.2% and 0.5% by weight.

6. A transdermal composition as in claim 1, wherein said gabapentin is between 7% and 12% by weight

7. A transdermal composition for the relief of pain in a subject comprising:
ketamine, clonidine and amitriptyline in a base.

8. A transdermal composition as in claim 7, wherein said base is pentravan gel.

9. A transdermal composition as in claim 7, wherein said base is 80% by weight pleurinic gel and 20% by weight lipoil.

10. A transdermal composition as in claim 7, wherein said ketamine is between 1% and 20% by weight.

11. A transdermal composition as in claim 7, wherein said clonidine is between 0.2 and 0.5% by weight.

12. A transdermal composition as in claim 7, wherein said amitriptyline is 1% to 5% by weight.

13. A transdermal composition for the relief of pain in a subject comprising:
ketamine and clonidine in a base.

14. A transdermal composition as in claim 13, wherein said base is pentravan gel.

15. A transdermal composition as in claim 13, wherein said base is 80% by weight pleurinic gel and 20% by weight lipoil.

16. A transdermal composition as in claim 13, wherein said ketamine is between 1% and 20% by weight.

17. A transdermal composition as in claim 13, wherein said clonidine is between 0.2% and 0.5% by weight.

18. A transdermal composition as in claim 13, further comprising ketoprofen.

19. A transdermal composition as in claim 18, wherein said ketoprofen is between 1% and 5% by weight.

20. A transdermal composition for the relief of pain in a subject comprising:
ketamine in a base.

21. A transdermal composition as in claim 20, wherein said base is pentravan gel.

22. A transdermal composition as in claim 20, wherein said base is 80% by weight pleurinic gel and 20% by weight lipoil.

23. A transdermal composition as in claim 20, wherein said ketamine is between 1% and 20% by weight.

24. A transdermal composition as in claim 20, further comprising clonidine.

25. A transdermal composition as in claim 24, wherein said clonidine is between 0.2% and 0.5% by weight.

26. A transdermal composition as in claim 20, further comprising ketoprofen.

27. A transdermal composition as in claim 20, wherein said ketoprofen is between 1% and 5% by weight.

28. A transdermal composition for the relief of pain in a subject comprising:
clonidine in a base.

29. A transdermal composition as in claim 28, wherein said base is pentravan gel.

30. A transdermal composition as in claim 28, wherein said base is 80% by weight pleurinic gel and 20% by weight lipoil.

31. A transdermal composition as in claim 28, wherein said clonidine is between 0.2% and 0.5% by weight.

32. A transdermal composition as in claim 28, further comprising ketamine.

33. A transdermal composition as in claim 32, wherein said ketamine is between 1% and 20% by weight.

34. A transdermal composition as in claim 28, further comprising ketoprofen.

35. A transdermal composition as in claim 34, wherein said ketoprofen is between 1% and 5% by weight.
